(19) United States
(12) Patent Application Publication
(10) Pub. No.: US 2003/0157119 A1
(43) Pub. Date: Aug. 21, 2003

(54) Methods for diagnosis and therapy of hematological and virus-associated malignancies

(75) Inventors: Alexander Gaiger, Seattle, WA (US); Martin A. Cheever, Mercer Island, WA (US); Susan Hand-Zimmermann, Redmond, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 10/313,644

(22) Filed: Dec. 4, 2002

Related U.S. Application Data
(63) Continuation of application No. 09/675,904, filed on Sep. 28, 2000, now abandoned.
Application No. 09/675,904 is a continuation-in-part of application No. 09/638,280, filed on Aug. 14, 2000, now abandoned.
Application No. 09/638,280 is a continuation-in-part of application No. 09/404,443, filed on Sep. 22, 1999, now abandoned.

(51) Int. Cl. A61K 48/00; A61K 39/00

(52) U.S. Cl. 424/185.1; 514/44; 424/277.1

(57) ABSTRACT

The present invention is directed to methods for detecting and treating hematological and virus-associated malignancies using Her2/neu sequences. The Her2/neu sequences may be polypeptides or polynucleotides.

### CROSS REFERENCE TO RELATED APPLICATIONS

This application is a CIP of U.S. application Ser. No. 09/638,280, which is CIP of U.S. application Ser. No. 09/404,443, and is related to a PCT application filed Aug. 22, 2000.

### TECHNICAL FIELD

The present invention relates generally to the therapy of malignancies, and more specifically to methods employing Her2/neu sequences for detection and therapy of hematological and virus-associated malignancies and lymphomas.

### BACKGROUND OF THE INVENTION

Hematological malignancies, such as leukemias, are neoplastic disorders of hematopoetic stem cells. Such diseases are characterized by abnormal growth and maturation of hematopoetic cells and can result in a variety of symptoms, including bone marrow failure and organ failure. Treatment for many hematological malignancies remains difficult and existing therapies are not universally effective. While treatments involving specific immunotherapy appear to have considerable potential, such treatments have been limited by the small number of known tumor-associated antigens.

Her-2/neu, the product of the Her2/neu oncogene (also known as p185 or c-erbB2; see, e.g., U.S. Pat. No. 5,869,445) is a self antigen that is known to be overexpressed in adenocarcinomas of the breast, ovary, colon and lung. The Her2/neu proto-oncogene encodes a tyrosine kinase with homology to epidermal growth factor. Her2/neu protein is expressed during fetal development, but in adults is detectable only in small amounts in a limited number of normal tissues. Her2/neu has been found to be expressed on leukemic blasts of some patients suffering from acute lymphatic leukemia (ALL; EP 771,565B1) and on malignant lymphoma cells of a patient afflicted with aggressive diffuse lymphoma (Imamura et al., Leukemia and Lymphoma 4:4129-422). Her2/neu has not, however, been detected on blasts from patients with acute myelogenous leukemia (AML) or chronic myelogenous leukemia (CML), in chronic or accelerated phase or in blast crisis. Thus, Her-2/neu has not appeared to be generally useful as a marker or therapeutic target for hematological malignancies.

Other prevalent malignancies that are often difficult to treat are virus-associated conditions, such as malignancies associated with Epstein Barr Virus (EBV) infection. Such malignancies include lymphomas in immunocompromised patients (e.g., AIDS patients and organ transplant recipients), nasopharynxcarcinoma and breast cancer. EBV-associated malignancies are common in immunocompromised individuals and are endemic in certain Asian populations. To date, there is no generally effective treatment for such conditions.

Accordingly, there remains a need in the art for improved methods for detecting and treating hematological and virus-associated malignancies. The present invention fulfills these needs and further provides other related advantages.

### SUMMARY OF THE INVENTION

Briefly stated, the present invention provides compositions and methods for detecting and treating hematological and virus-associated malignancies. Within certain aspects, the present invention provides methods for inhibiting the development of a hematological malignancy or virus-associated malignancy in a patient. Such methods may comprise administering to a patient an effective amount of a polypeptide comprising at least an immunogenic portion of Her2/neu, or a variant thereof that differs only in conservative substitutions such that the immunogenicity of the variant is not substantially diminished. Alternatively, a polynucleotide encoding such a polypeptide, and antigen-presenting cell expressing such a polypeptide, or an antibody, or antigen-binding fragment thereof that specifically binds Her2/neu may be administered. Hematological malignancies include AML, CML, CLL, MDS, myelomas, Hodgkin lymphomas and non-Hodgkin lymphomas. Virus-associated malignancies include malignancies associated with EBV, cytomegalovirus or adenovirus, such as lymphomas and nasopharynxcarcinoma.

Within further aspects, methods for inhibiting the development of a hematological or virus-associated malignancy in a patient comprise the steps of: (a) incubating CD4⁺ and/or CD8+ T cells isolated from a patient with at least one component selected from the group consisting of: (i) a polypeptide comprising at least an immunogenic portion of Her2/neu; (ii) a polynucleotide encoding such a polypeptide; and (iii) an antigen-presenting cell that expresses such a polypeptide; such that T cells proliferate; and (b) administering to the patient an effective amount of the proliferated T cells. Proliferated T cells may, but need not, be cloned prior to administration to the patient. The patient may be afflicted with a hematological or virus-associated malignancy, in which case the methods provide treatment for the disease, or a patient considered at risk for the disease may be treated prophylactically.

The present invention further provides methods for determining the presence or absence of a hematological or virus-associated malignancy in a patient, comprising the steps of: (a) contacting a biological sample obtained from a patient with a binding agent that specifically binds to Her2/neu; (b) detecting in the sample an amount of polypeptide that binds to the binding agent; and (c) comparing the amount of polypeptide to a predetermined cut-off value.

Within related aspects, methods are provided for monitoring the progression of a hematological or virus-associated malignancy in a patient, comprising the steps of: (a) contacting a biological sample obtained from a patient at a first point in time with a binding agent that specifically binds to Her2/neu; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) to the amount detected in step (b).

The present invention provides, within further aspects, methods for determining the presence or absence of a hematological or virus-associated malignancy in a patient, comprising the steps of: (a) contacting a biological sample obtained from a patient with an oligonucleotide that hybridizes to a polynucleotide that encodes Her2/neu; (b) detecting in the sample an amount of a polynucleotide that hybridizes to the oligonucleotide; and (c) comparing the amount of polynucleotide to a predetermined cut-off value.

In related aspects, methods are provided for monitoring the progression of a hematological or virus-associated malignancy in a patient, comprising the steps of: (a) contacting a biological sample obtained from a patient with an oligonucleotide that hybridizes to a polynucleotide that encodes Her2/neu; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) to the amount detected in step (b).

Methods are further provided for inhibiting the development of a hematological or virus-associated malignancy in a patient, comprising: (a) contacting bone marrow, peripheral blood, or a fraction of bone marrow or peripheral blood with T cells that specifically react with Her2/neu, wherein the step of contacting is performed under conditions and for a time sufficient to permit the removal of Her2/neu positive cells to less than 10% of the number of myeloid or lymphatic cells in the bone marrow, peripheral blood or a fraction of bone marrow or peripheral blood; and (b) administering to a patient the bone marrow, peripheral blood or fraction from which Her2/neu positive cells have been removed.

In certain illustrative embodiments, an immunogenic portion of Her2/neu that is employed in the practice of the present invention comprises an amino acid sequence consisting essentially of a naturally processed, Her2/neu CTL epitope set forth in SEQ ID NO: 3.

These and other aspects of the invention will become evident upon reference to the following detailed description and attached drawings. All references disclosed herein are hereby incorporated by reference in their entirety as if each were individually noted for incorporation.

### BRIEF DESCRIPTION OF THE DRAWINGS

FIGS. 1A-1D are graphs illustrating the results of FACS staining for Her2/. FIG. 1A shows FACS staining of the Her2/neu overexpressing breast cancer cell line SKBR3, FIG. 1B shows FACS staining of human lymphoma cells, FIG. 1C shows staining of human AML cells and FIG. 1D shows staining of human CLL cells using a control antibody (negative control; dotted line) and a Her2/neu ECD specific antibody (solid line). Abbreviations: MFI=mean fluorescence intensity; % positive=% of cells staining positive for Her2/neu.

FIG. 2 is a graph depicting the results of 51Cr-release assays demonstrating ICD reactivity in a CD8+ T cell line primed with AdV. Normal donor PBMC were primed with DC-infected with recombinant AdV expressing ICD. The assay was a standard 4 hour ⁵¹Cr-release assay; targets were autologous B-LCL, either uninfected or infected with recombinant cavvinia virus expessing ICD oe EGFP, as indicated. Each data point was the average of three measurements.

FIG. 3 is a graph depicting the results of flow cytometric analysis of surface Her-2/neu on MCF-7 tumor cells. Cells were stained with a mAb to surface Her-2/neu, followed by a secondary rabbit anti-mouse Ig antibody conjugated to PE. Labeled cells were analyzed by flow cytometry. Values for mean fluorescent intensity were as follows: MCF-7=32; MCF-7+RTV-H2N=165; MCF-7+Ad-H2N=683; MCF-7+RTV-H2N+Ad-H2N=651.

### DETAILED DESCRIPTION OF THE INVENTION

As noted above, the present invention provides methods for detecting and treating hematological and virus-associated malignancies. The invention is based, in part, on the discovery that Her2/neu is overexpressed in patients with hematological malignancies, as well as in EBV-transduced B-cells and EBV-induced lymphomas. It has further been found, within the context of the present invention, that inoculating SCID mice with EBV-infected B-cells induces lymphomas that overexpress Her2/neu in all animals. Vaccination with Her2/neu may be effective in preventing and/or treating hematological malignancies, including adult and pediatric AML, CML, ALL, CLL, myelodysplastic syndromes (MDS), myeloproliferative syndromes (MPS), secondary leukemia, myeloma, Hodgkin lymphoma and Non-Hodgkin lymphoma. Such vaccination may also be used to prevent and/or treat virus-associated malignancies, such as conditions resulting from EBV infection. Alternatively, antibody therapy may be used for treatment and/or prevention of hematological and virus-associated malignancies. Further, Her2/neu expression may be used for the diagnosis of such malignancies, monitoring therapy and purging bone marrow for transplantation.

Her-2/neu Polypeptides

Certain methods provided herein employ Her-2/neu polypeptides. Such polypeptides may comprise at least an immunogenic portion of a native Her2/neu protein. Alternatively, a Her-2/neu polypeptide may comprise a variant of such a portion that differs only in conservative substitutions such that the immunogenicity of the variant is not substantially diminished, relative to the native immunogenic portion.

As noted above, Her2/neu is the product of the Her2/neu oncogene, also known as p185 or c-erbB2 (see, e.g., U.S. Pat. No. 5,869,445). “Her2/neu,” as used herein refers to published Her2/neu sequences, including human sequence, alleles thereof and homologs from other species.

An “immunogenic portion,” as used herein is a portion of a protein that is recognized (i.e., specifically bound) by a B-cell and/or T-cell surface antigen receptor. Such immunogenic portions generally comprise at least 5 amino acid residues, more preferably at least 10, and still more preferably at least 20 amino acid residues of Her2/neu. Certain preferred immunogenic portions include peptides in which an N-terminal leader sequence and/or transmembrane domain have been deleted. Other preferred immunogenic portions may contain a small N- and/or C-terminal deletion (e.g., 1-30 amino acids, preferably 5-15 amino acids), relative to the mature protein. Preferred immunogenic portions are derived from the extracellular domain of Her2/neu. The sequence of a human Her2/neu protein and certain immunogenic portions thereof are provided within, for example, U.S. Pat. No. 5,726,023.

Immunogenic portions may generally be identified using well known techniques, such as those summarized in Paul, Fundamental Immunology, 3rd ed., 243-247 (Raven Press, 1993) and references cited therein. Such techniques include screening portions of Her2/neu for the ability to react with Her2/neu-specific antibodies, antisera and/or T-cell lines or clones. As used herein, antisera and antibodies are “Her2/neu-specific” if they specifically bind to Her2/neu (i.e., they react with the protein in an ELISA or other immunoassay, and do not react detectably with unrelated proteins). Such antisera and antibodies may be prepared as described herein, and using well known techniques. An immunogenic portion of a native Her2/neu is a portion of Her2/neu that reacts with such antisera and/or T-cells at a level that is not substantially less than the reactivity of the full length polypeptide (e.g., in an ELISA and/or T-cell reactivity assay). Such immunogenic portions may react within such assays at a level that is similar to or greater than the reactivity of a full length Her2/neu. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

A “variant” of an immunogenic portion is a polypeptide that differs from a native immunogenic portion of Her2/neu in one or more substitutions, deletions and/or insertions, such that the immunogenicity of the polypeptide is not substantially diminished. In other words, the ability of a variant to react with antigen-specific antisera may be enhanced or unchanged, relative to the native immunogenic portion, or may be diminished by less than 50%, and preferably less than 20%, relative to the native protein. Such variants may generally be identified by modifying an immunogenic portion and evaluating the reactivity of the modified polypeptide with antigen-specific antibodies or antisera as described herein.

Variants preferably exhibit at least about 70%, more preferably at least about 90% and most preferably at least about 95% identity to a native immunogenic portion. The percent identity may be determined as described above. Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or insertion of five amino acids or fewer.

As noted above, a Her2/neu polypeptide may contain sequences in addition to the immunogenic portion or variant thereof. Such sequences may, but need not, be derived from Her-2/neu. Sequences that are not derived from Her-2/neu may, but need not, be present at the amino and/or carboxy terminus of the polypeptide. Such sequence(s) may be used, for example, to facilitate synthesis, purification or solubilization. Other sequences that may be present include, but are not limited to, a signal (or leader) sequence at the N-terminal end of the protein which co-translationally or post-translationally directs transfer of the protein.

Polypeptides may be prepared using any of a variety of well known techniques. Recombinant polypeptides encoded by Her2/neu DNA sequences as described herein may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having fewer than about 100 amino acids, and generally fewer than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions. Crude product can be further purified by gel filtration, HPLC, partition chromatography or ion-exchange chromatography, using well known procedures.

Within certain specific embodiments, a polypeptide may be a fusion protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Fusion proteins are also provided that comprise a polypeptide as described herein together with an unrelated immunogenic protein. Preferably, the immunogenic protein is capable of eliciting a recall response. Examples of such proteins include tetanus, tuberculosis and hepatitis proteins (see, e.g., Stoute et al., New Engl. J. Med. 336:86-91, 1997).

Within preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen present cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coil C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Her2/neu Polynucleotides

Any polynucleotide that encodes at least a portion of a Her2/neu polypeptide as described herein is encompassed by the present invention. Preferred polynucleotides comprise at least 15 consecutive nucleotides, preferably at least 30 consecutive nucleotides and more preferably at least 45 consecutive nucleotides, that encode a portion of Her2/neu. Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes Her2/neu or a portion thereof) or may comprise a variant of such a sequence. Polynucleotide variants may contain one or more substitutions, additions, deletions and/or insertions such that the immunogenicity of the encoded polypeptide is not diminished, relative to native Her2/neu. The effect on the immunogenicity of the encoded polypeptide may generally be assessed as described herein. Variants preferably exhibit at least about 70% identity, more preferably at least about 80% identity and most preferably at least about 90% identity to a polynucleotide sequence that encodes a native Her2/neu or a portion thereof.

The percent identity for two polynucleotide or polypeptide sequences may be readily determined by comparing sequences using computer algorithms well known to those of ordinary skill in the art, such as Megalign, using default parameters. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned. Optimal alignment of sequences for comparison may be conducted, for example, using the Megalign program in the Lasergene suite of bioinformatics software (DNASTAR, Inc., Madison, Wis.), using default parameters. Preferably, the percentage of sequence identity is determined by comparing two optimally aligned sequences over a window of comparison of at least 20 positions, wherein the portion of the polynucleotide or polypeptide sequence in the window may comprise additions or deletions (i.e., gaps) of 20% or less, usually 5 to 15%, or 10 to 12%, relative to the reference sequence (which does not contain additions or deletions). The percent identity may be calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native Her2/neu (or a complementary sequence). Suitable moderately stringent conditions include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention.

Polynucleotides may be prepared using any of a variety of techniques. 94:2150-2155, 1997). For example, polynucleotides may be amplified from cDNA prepared from cells expressing Her2/neu, such as certain breast tumor cells. Such polynucleotides may be amplified via polymerase chain reaction (PCR). For this approach, sequence-specific primers may be designed based on known sequences, and may be purchased or synthesized.

An amplified portion may be used to isolate a full length gene from a suitable library (e.g., a breast tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences are then assembled into a single contiguous sequence. A full length cDNA molecule can be generated by ligating suitable fragments, using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

Polynucleotide variants may generally be prepared by any method known in the art, including chemical synthesis by, for example, solid phase phosphoramidite chemical synthesis. Modifications in a polynucleotide sequence may also be introduced using standard mutagenesis techniques, such as oligonucleotide-directed site-specific mutagenesis (see Adelman et al., DNA 2:183, 1983). Alternatively, RNA molecules may be generated by in vitro or in vivo transcription of DNA sequences, provided that the DNA is incorporated into a vector with a suitable RNA polymerase promoter (such as T7 or SP6). Certain portions may be used to prepare an encoded polypeptide, as described herein. In addition, or alternatively, a portion may be administered to a patient such that the encoded polypeptide is generated in vivo (e.g., by transfecting antigen-presenting cells, such as dendritic cells, with a cDNA construct encoding a Her2/neu, and administering the transfected cells to the patient).

A portion of a sequence complementary to a coding sequence (i.e., an antisense polynucleotide) may also be used as a probe or to modulate Her2/neu expression. cDNA constructs that can be transcribed into antisense RNA may also be introduced into cells or tissues to facilitate the production of antisense RNA. An antisense polynucleotide may be used, as described herein, to inhibit expression of a Her2/neu. Antisense technology can be used to control gene expression through triple-helix formation, which compromises the ability of the double helix to open sufficiently for the binding of polymerases, transcription factors or regulatory molecules (see Gee et al., In Huber and Carr, Molecular and Immunologic Approaches, Futura Publishing Co. (Mt. Kisco, N.Y.; 1994)). Alternatively, an antisense molecule may be designed to hybridize with a control region of a gene (e.g., promoter, enhancer or transcription initiation site), and block transcription of the gene; or to block translation by inhibiting binding of a transcript to ribosomes.

A portion of a coding sequence or of a complementary sequence may also be designed as a probe or primer to detect gene expression. Probes may be labeled with a variety of reporter groups, such as radionuclides and enzymes, and are preferably at least 10 nucleotides in length, more preferably at least 20 nucleotides in length and still more preferably at least 30 nucleotides in length. Primers, as noted above, are preferably 22-30 nucleotides in length.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Her2/neu polynucleotides may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Binding Agents

The present invention further employs agents, such as antibodies and antigen-binding fragments thereof, that specifically bind to Her2/neu. As used herein, an antibody, or antigen-binding fragment thereof, is said to “specifically bind” to Her2/neu if it reacts at a detectable level (within, for example, an ELISA) with Her2/neu, and does not react detectably with unrelated proteins under similar conditions. As used herein, “binding” refers to a noncovalent association between two separate molecules such that a complex is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant maybe determined using methods well known in the art.

Binding agents may be further capable of differentiating between patients with and without a hematological malignancy. Such binding agents generate a signal indicating the presence of a hematological malignancy in at least about 20% of patients with the disease, and will generate a negative signal indicating the absence of the disease in at least about 90% of individuals without the disease. To determine whether a binding agent satisfies this requirement, biological samples (e.g., blood, sera, urine and/or tumor biopsies) from patients with and without a hematological malignancy (as determined using standard clinical tests) may be assayed as described herein for the presence of polypeptides that bind to the binding agent. It will be apparent that a statistically significant number of samples with and without the disease should be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

Monoclonal antibodies, and fragments thereof, of the present invention may be coupled to one or more therapeutic agents, such as radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein. For certain in vivo and ex vivo therapies, an antibody or fragment thereof is preferably coupled to a cytotoxic agent, such as a radioactive or chemotherapeutic moiety.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers which provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/immunoconjugate will vary depending upon the antibody used, the antigen density on the tumor, and the rate of clearance of the antibody.

T Cells

Immunotherapeutic compositions may also, or alternatively, comprise T cells specific for Her2/neu. Such cells may generally be prepared in vitro or ex vivo, using standard procedures. For example, T cells may be isolated from bone marrow, peripheral blood or a fraction of bone marrow or peripheral blood of a patient, using a commercially available cell separation system, such as the CEPRATE™ system, available from CellPro Inc., Bothell Wash. (see also U.S. Pat. Nos. 5,240,856; 5,215,926; WO 89/06280; WO 91/16116 and WO 92/07243). Alternatively, T cells may be derived from related or unrelated humans, non-human mammals, cell lines or cultures.

T cells may be stimulated with a Her2/neu polypeptide, Her2/neu polynucleotide and/or an antigen presenting cell (APC) that expresses a Her2/neu polypeptide. Such stimulation is performed under conditions and for a time sufficient to permit the generation of T cells that are specific for the polypeptide. Preferably, a Her2/neu polypeptide or polynucleotide is present within a delivery vehicle, such as a microsphere, to facilitate the generation of specific T cells.

T cells are considered to be specific for Her2/neu if the T cells kill target cells coated with Her2/neu or expressing a gene encoding Her2/neu. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a Her2/neu polypeptide (100 ng/ml -100 μg/ml, preferably 200 ng/ml -25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a Her2/neu polypeptide, polynucleotide or polypeptide-expressing APC may be CD4⁺ and/or CD8⁺. Her2/neu-specific T cells may be expanded using standard techniques. Within preferred embodiments, the T cells are derived from a patient, or from a related or unrelated donor, and are administered to the patient following stimulation and expansion.

For therapeutic purposes, CD4⁺ or CD8⁺ T cells that proliferate in response to a Her2/neu polypeptide, polynucleotide or APC can be expanded in number either in vitro or in vivo. Proliferation of such T cells in vitro may be accomplished in a variety of ways. For example, the T cells can be re-exposed to a Her2/neu polypeptide (e.g., a short peptide corresponding to an immunogenic portion of Her2/neu) with or without the addition of T cell growth factors, such as interleukin-2, and/or stimulator cells that synthesize a Her2/neu polypeptide. Alternatively, one or more T cells that proliferate in the presence of Her2/neu can be expanded in number by cloning. Methods for cloning cells are well known in the art, and include limiting dilution. Following expansion, the cells may be administered back to the patient as described, for example, by Chang et al., Crit. Rev. Oncol. Hematol. 22:213, 1996.

Pharmaceutical Compositions and Vaccines

Within certain aspects, polypeptides, polynucleotides, T cells and/or binding agents described herein may be incorporated into pharmaceutical compositions or immunogenic compositions (i.e., vaccines). Pharmaceutical compositions comprise one or more such compounds and a physiologically acceptable carrier. Vaccines may comprise one or more such compounds and a non-specific immune response enhancer. A non-specific immune response enhancer may be any substance that enhances an immune response to an exogenous antigen. Examples of non-specific immune response enhancers include adjuvants, biodegradable microspheres (e.g., polylactic galactide) and liposomes (into which the compound is incorporated; see e.g., Fullerton, U.S. Pat. No. 4,235,877). Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive.

A pharmaceutical composition or vaccine may contain a polynucleotide (DNA or RNA) encoding one or more of the polypeptides as described above, such that the polypeptide is generated in situ. As noted above, a polynucleotide may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

Any of a variety of non-specific immune response enhancers may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, IL-2 and IL-12) tend to favor the induction of cell mediated immune responses to an administered antigen. In contrast, high levels of Th2-type cytokines (e.g., IL-4, IL-5, IL-6, IL-10 and TNF-β) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffinan, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Ribi ImmunoChem Research Inc. (Hamilton, Mont.; see U.S. Pat. Nos. 4,436,727; 4,877,611; 4,866,034 and 4,912,094). Also preferred is AS-2 (SmithKline Beecham). CpG-containing oligonucleotides (in which the CpG dinucleotide is unmethylated) also induce a predominantly Th1 response. Such oligonucleotides are well known and are described, for example, in WO 96/02555. Another preferred adjuvant is a saponin, preferably QS21, which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprises an oil-in-water emulsion and tocopherol. A particularly potent adjuvant formulation involving QS21, 3D-MPL and tocopherol in an oil-in-water emulsion is described in WO 95/17210. Any vaccine provided herein may be prepared using well known methods that result in a combination of antigen, immune response enhancer and a suitable carrier or excipient.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro) and based on the lack of differentiation markers of B cells (CD19 and CD20), T cells (CD3), monocytes (CD14) and natural killer cells (CD56), as determined using standard assays. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce maturation and proliferation of dendritic cells. Dendritic cells may alternatively be generated from leukemic and lymphoma cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor, mannose receptor and DEC-205 marker. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80 and CD86).

APCs may generally be transfected with a polynucleotide encoding a Her2/neu polypeptide such that the polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the Her2/neu polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Therapeutic Methods

In further aspects of the present invention, the compositions described herein may be used for immunotherapy of hematological malignancies including adult and pediatric AML, CML, ALL, CLL, myelodysplastic syndromes (MDS), myeloproliferative syndromes (MPS), secondary leukemia, multiple myeloma, Hodgkin lymphoma and Non-Hodgkin lymphomas. Such compositions may further be used for immunotherapy of virus-associated malignancies (i.e., malignancies in which viral infection is detectable). Viruses that may be associated with malignancies include, but are not limited to, EBV, adenovirus and cytomegalovirus. EBV-associated malignancies include, for example, lymphomas and nasopharynxcarcinoma. Certain EBV-associated malignancies are post-transplant lymphomas (e.g., following transplant of an organ such as liver, heart, kidney or bone marrow) and lymphomas in immunocompromised patients (such as AIDS patients). In addition, compositions described herein may be used for therapy of diseases associated with an autoimmune response against hematopoetic precursor cells, such as leukopenia and pancytopenia (e.g., severe aplastic anemia). In particular, such therapies may effectively treat or prevent such diseases caused by immunity to Her2/neu (i.e., the presence of antibodies to Her2/neu that induce apoptosis in hematopoetic precursor cells).

Immunotherapy may be performed using any of a variety of techniques, in which compounds or cells provided herein function to remove Her2/neu-expressing cells from a patient. Such removal may take place as a result of enhancing or inducing an immune response in a patient specific for Her2/neu or a cell expressing Her2/neu. Alternatively, Her2/neu-expressing cells may be removed ex vivo (e.g., by treatment of autologous bone marrow, peripheral blood or a fraction of bone marrow or peripheral blood). Fractions of bone marrow or peripheral blood may be obtained using any standard technique in the art.

Within such methods, pharmaceutical compositions and vaccines are typically administered to a patient. As used herein, a “patient” refers to any warm-blooded animal, preferably a human. A patient may or may not be afflicted with a hematological malignancy or virus-associated malignancy. Accordingly, the above pharmaceutical compositions and vaccines may be used to prevent the development of a malignancy or to treat a patient afflicted with a malignancy. A hematological malignancy or virus-associated malignancy may be diagnosed using criteria generally accepted in the art. Pharmaceutical compositions and vaccines may be administered either prior to or following surgical removal of primary tumors and/or treatment such as administration of radiotherapy or conventional chemotherapeutic drugs, or bone marrow transplantation (autologous, allogeneic or syngeneic).

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

The compositions provided herein may be used alone or in combination with conventional therapeutic regimens such as surgery, irradiation, chemotherapy and/or bone marrow transplantation (autologous, syngeneic, allogeneic or unrelated). As discussed in greater detail below, binding agents and T cells as provided herein may be used for purging of autologous stem cells. Such purging may be beneficial prior to, for example, bone marrow transplantation or transfusion of blood or components thereof. Binding agents, T cells, antigen presenting cells (APC) and compositions provided herein may further be used for expanding and stimulating (or priming) autologous, allogeneic, syngeneic or unrelated Her2/neu-specific T-cells in vitro and/or in vivo. Such Her2/neu-specific T cells may be used, for example, within donor lymphocyte infusions.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 100 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to Her2/neu generally correlate with an improved clinical outcome. Such immune responses may generally be evaluated using standard proliferation, cytotoxicity or cytokine assays, which may be performed using samples obtained from a patient before and after treatment.

Within further aspects, methods for inhibiting the development of a malignant disease associated with Her2/neu expression involve the administration of autologous T cells that have been activated in response to a Her2/neu polypeptide or Her2/neu-expressing APC, as described above. Such T cells may be CD4⁺ and/or CD8⁺, and may be proliferated as described above. The T cells may be administered to the individual in an amount effective to inhibit the development of a malignant disease. Typically, about 1×10⁹ to 1×10¹¹ T cells/M² are administered intravenously, intracavitary or in the bed of a resected tumor. It will be evident to those skilled in the art that the number of cells and the frequency of administration will be dependent upon the response of the patient.

Within certain embodiments, T cells may be stimulated prior to an autologous bone marrow transplantation. Such stimulation may take place in vivo or in vitro. For in vitro stimulation, bone marrow and/or peripheral blood (or a fraction of bone marrow or peripheral blood) obtained from a patient may be contacted with a Her2/neu polypeptide, a polynucleotide encoding a Her2/neu polypeptide and/or an APC that expresses a Her2/neu polypeptide under conditions and for a time sufficient to permit the stimulation of T cells as described above. Bone marrow, peripheral blood stem cells and/or Her2/neu-specific T cells may then be administered to a patient using standard techniques.

Within related embodiments, T cells of a related or unrelated donor may be stimulated prior to a syngeneic or allogeneic (related or unrelated) bone marrow transplantation. Such stimulation may take place in vivo or in vitro. For in vitro stimulation, bone marrow and/or peripheral blood (or a fraction of bone marrow or peripheral blood) obtained from a related or unrelated donor may be contacted with a Her2/neu polypeptide, Her2/neu polynucleotide and/or APC that expresses a Her2/neu polypeptide under conditions and for a time sufficient to permit the stimulation of T cells as described above. Bone marrow, peripheral blood stem cells and/or Her2/neu-specific T cells may then be administered to a patient using standard techniques.

Within other embodiments, Her2/neu-specific T cells as described herein may be used to remove cells expressing Her2/neu from autologous bone marrow, peripheral blood or a fraction of bone marrow or peripheral blood (e.g., CD34⁺ enriched peripheral blood (PB) prior to administration to a patient). Such methods may be performed by contacting bone marrow or PB with such T cells under conditions and for a time sufficient to permit the reduction of Her2/neu expressing cells to less than 10%, preferably less than 5% and more preferably less than 1%, of the total number of myeloid or lymphatic cells in the bone marrow or peripheral blood. The extent to which such cells have been removed may be readily determined by standard methods such as, for example, qualitative and quantitative PCR analysis, morphology, immunohistochemistry and FACS analysis. Bone marrow or PB (or a fraction thereof) may then be administered to a patient using standard techniques.

Diagnostic Methods

In general, a hematological or virus-associated malignancy may be detected in a patient based on the presence of Her2/neu protein and/or polynucleotide in a biological sample (such as blood, sera, urine and/or tumor biopsies) obtained from the patient. In other words, Her2/neu may be used as a marker to indicate the presence or absence of such a malignancy. The binding agents provided herein generally permit detection of the level of antigen that binds to the agent in the biological sample. Polynucleotide primers and probes may be used to detect the level of mRNA encoding Her2/neu, which is also indicative of the presence or absence of a hematological or virus-associated malignancy. In general, Her2/neu sequence should be present at a level that is at least three fold higher in a sample obtained from a patient afflicted with a hematological or virus-associated malignancy than in the sample obtained from an individual not so afflicted.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a hematological malignancy in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length Her2/neu and portions thereof to which the binding agent binds, as described above.

The solid support may be any material known to those of ordinary skill in the art to which the Her2/neu polypeptide may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent maybe immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with a hematological malignancy. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a hematological malignancy, the signal detected from the reporter group that remains bound to the solid support is generally compared to a signal that corresponds to a predetermined cut-off value. In one preferred embodiment, the cut-off value for the detection of a hematological or virus-associated malignancy is the average mean signal obtained when the immobilized antibody is incubated with samples from patients without the malignancy. In general, a sample generating a signal that is three standard deviations above the predetermined cut-off value is considered positive for the malignancy. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a malignancy.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a hematological or virus-associated malignancy. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the Her2/neu sequences or binding agents of the present invention. The above descriptions are intended to be exemplary only. For example, it will be apparent to those of ordinary skill in the art that the above protocols may be readily modified to use Her2/neu polypeptides to detect antibodies that bind to such polypeptides in a biological sample. The detection of Her2/neu-specific antibodies may correlate with the presence of a hematological or virus-associated malignancy.

A malignancy may also, or alternatively, be detected based on the presence of T cells that specifically react with Her2/neu in a biological sample. Within certain methods, a biological sample comprising CD4⁺ and/or CD8⁺ T cells isolated from a patient is incubated with a Her2/neu polypeptide, a polynucleotide encoding such a polypeptide and/or an APC that expresses such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with Mtb-81 or Mtb-67.2 polypeptide (e.g., 5-25 μg/ml). It may be desirable to incubate another aliquot of a T cell sample in the absence of Her2/neu polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a hematological or virus-associated malignancy in the patient.

As noted above, a hematological or virus-associated malignancy may also, or alternatively, be detected based on the level of mRNA encoding Her2/neu in a biological sample. For example, at least two oligonucleotide primers may be employed in a polymerase chain reaction (PCR) based assay to amplify a portion of Her2/neu cDNA derived from a biological sample, wherein at least one of the oligonucleotide primers is specific for (i.e., hybridizes to) a polynucleotide encoding the Her2/neu protein. The amplified cDNA is then separated and detected using techniques well known in the art, such as gel electrophoresis. Similarly, oligonucleotide probes that specifically hybridize to a polynucleotide encoding Her2/neu may be used in a hybridization assay to detect the presence of polynucleotide encoding Her2/neu in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding Her2/neu that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample such as a biopsy tissue and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a hematological or virus-associated malignancy. The amplification reaction may be performed on several dilutions of cDNA spanning two orders of magnitude. A two-fold or greater increase in expression in several dilutions of the test patient sample as compared to the same dilutions of the sample from a normal individual is typically considered positive.

In another embodiment, Her2/neu may be used as a marker for monitoring the progression or therapy of a hematological or virus-associated malignancy. In this embodiment, assays as described above for the diagnosis of a hematological or virus-associated malignancy may be performed over time, and the change in the level of reactive polypeptide(s) evaluated. For example, the assays may be performed every 24-72 hours for a period of 6 months to 1 year, and thereafter performed as needed. In general, a malignancy is progressing in those patients in whom the level of polypeptide detected by the binding agent increases over time. In contrast, the malignancy is not progressing when the level of reactive polypeptide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple markers may be assayed within a given sample. It will be apparent that binding agents specific for different proteins provided herein may be combined within a single assay. Further, multiple primers or probes may be used concurrently. The selection of markers may be based on routine experiments to determine combinations that results in optimal sensitivity.

Further diagnostic applications include the detection of extramedullary disease (e.g., cerebral infiltration of blasts in leukemias). Within such methods, a binding agent may be coupled to a tracer substance, and the diagnosis is performed in vivo using well known techniques. Coupled binding agent may be administered as described above, and extramedullary disease may be detected based on assaying the presence of tracer substance. Alternatively, a tracer substance may be associated with a T cell specific for Her2/neu, permitting detection of extramedullary disease based on assays to detect the location of the tracer substance.

Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to Her2/neu. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding Her2/neu in a biological sample. Such kits generally comprise at least one oligonucleotide probe or primer, as described above, that hybridizes to a polynucleotide encoding Her2/neu. Such an oligonucleotide may be used, for example, within a PCR or hybridization assay. Additional components that may be present within such kits include a second oligonucleotide and/or a diagnostic reagent or container to facilitate the detection of a polynucleotide encoding Her2/neu.

The following Examples are offered by way of illustration and not by way of limitation.

### EXAMPLES

### Example 1

### Her2/neu Expression in Leukemia Patients

This Example illustrates the use of real time PCR analysis to identify Her2/neu as a marker for hematological malignancies.

A leukemia cDNA panel was generated from mononuclear cells isolated from bone marrow aspirates and peripheral blood samples using Lymphoprep (Nycomed, Oslo, Norway). RNA was extracted according to standard protocols. To assess the purity of mRNA, all RNA samples were analyzed on denaturing formamide agarose gels and control amplification of beta-actin cDNA was performed. 2 μg of RNA was used for reverse transcription according to standard protocols.

This panel was analyzed using real time PCR to compare the relative level of Her2/neu mRNA in bone marrow and peripheral blood of leukemia patients (AML and CML) with normal peripheral blood and bone marrow. The real time PCR analysis showed overexpression of Her2/neu mRNA in 33% of leukemia patients, relative to normal peripheral blood and bone marrow. Results are presented in Table 1.

TABLE 1
Real Time PCR Results

| | Her2neu | | |
|---|---|---|---|
| Tissue Type | Replicate 1 | Replicate 2 | Mean |
| Human Cell Line | 1056.500 | 1051.900 | 1054.200 |
| Human Cell Line | 88.225 | 22.914 | 55.570 |
| Human Cell Line | 4466.600 | 2070.300 | 3268.450 |
| CML | 300.300 | 245.740 | 273.020 |
| CML | 42.688 | 123.070 | 82.879 |
| AML | 10.948 | 12.681 | 11.815 |
| AML | 84.732 | 105.420 | 95.076 |
| AML | 78.452 | 214.420 | 146.436 |
| AML | 93.151 | 25.111 | 59.131 |
| AML | 72.619 | 103.450 | 88.035 |
| AML | 33.164 | 12.790 | 22.977 |
| AML | 652.650 | 221.860 | 435.255 |
| AML | 7530.000 | 7065.400 | 7297.700 |
| AML | 307.210 | 1739.300 | 1023.255 |
| AML | 319.710 | 462.850 | 391.280 |
| AML | 77.453 | 118.680 | 98.067 |
| AML | 99.729 | 49.020 | 74.375 |
| AML | 145.360 | 81.941 | 113.651 |
| Bone Marrow Normal | 145.740 | 118.990 | 132.365 |
| Bone Marrow Normal | 107.110 | 267.250 | 187.180 |
| Bone Marrow Normal | 384.270 | 382.110 | 383.190 |
| Whole Blood 1 | 0 | 0 | 0 |
| Whole Blood 2 | 0 | 0 | 0 |
| Whole Blood 3 | 0 | 0 | 0 |
| Whole Blood 4 | 0 | 0 | 0 |
| Whole Blood 5 | 0 | 0 | 0 |
| Whole Blood 6 | 0 | 0 | 0 |
| Whole Blood 7 | 0 | 0 | 0 |
| Whole Blood 8 | 0 | 0 | 0 |
| Whole Blood 9 | 0 | 0 | 0 |
| Whole Blood 10 | 0 | 0 | 0 |
| Whole Blood 11 | 0 | 0 | 0 |
| Whole Blood 12 | 0 | 0 | 0 |
| Whole Blood 13 | 0 | 0 | 0 |
| Whole Blood 14 | 0 | 0 | 0 |
| Whole Blood 15 | 0 | 0 | 0 |

### Example 2

### Her2/neu Immune Responses in Leukemia Patients

This Example illustrates the detection of preexisting antibody and T cell responses in leukemia patients, and the examination of Her2/neu expression in leukemic cells using FACS analysis.

For Her2/neu staining, cells were stained in buffer consisting of 10% FCS (fetal calf serum) in HBSS (Hank's balanced salt solution) containing 0.1% NaN3. Cells were incubated for 20 minutes on ice with 10 μg/ml biotin-conjugated Herceptin (Genentech) or control as the primary step, followed by PE-streptavidin (1:000 dilution, Fisher) for detection. Cells were then washed twice between staining steps with staining buffer and resuspended in PBS prior to analysis. Cells were analyzed by flow cytometry using a Becton Dickinson FACSCalibur instrument using standard techniques. Ten thousand events were collected for analysis.

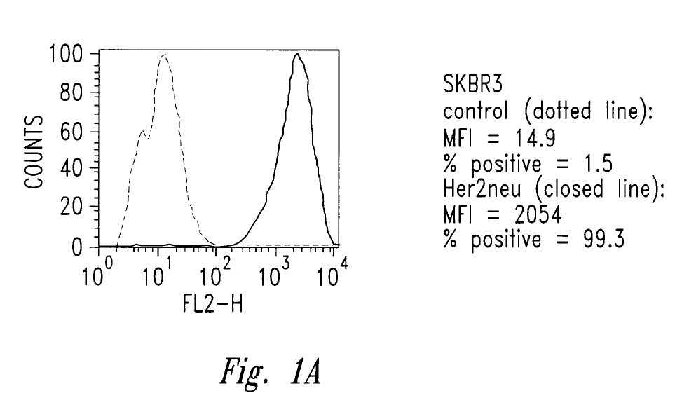

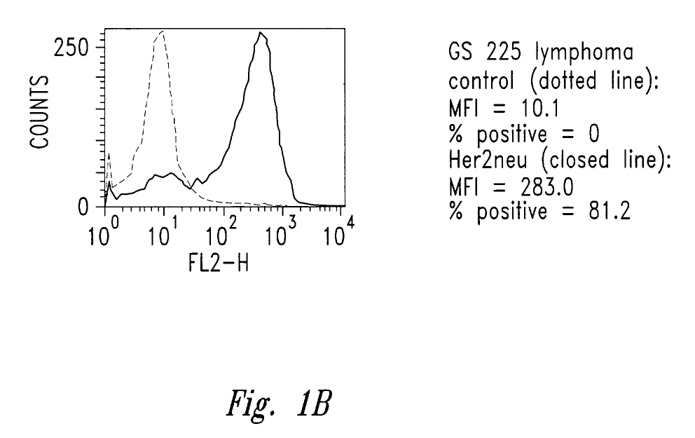

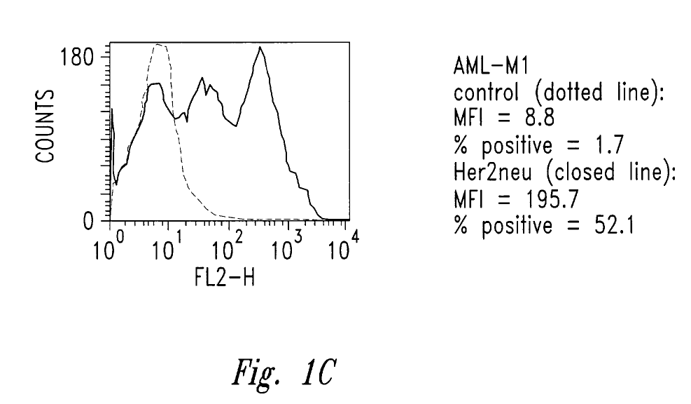

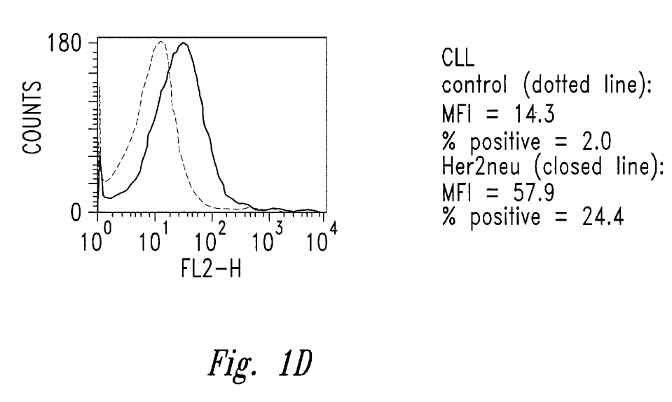

Results of FACS staining for Her2/neu are presented in FIGS. 1A-1D. FIG. 1A shows FACS staining of the Her2/neu overexpressing breast cancer cell line SKBR3. Also shown are staining of human lymphoma cells (FIG. 1B), human AML cells (FIG. 1C) and human CLL cells (FIG. 1D) using a control antibody (negative control) and a Her2/neu ECD specific antibody. FACS analyzes showed Her2/neu overexpression in the positive control SKBR3, as well as the clinical samples of a lymphoma, AML and CLL patient.

The human leukemia cell line K562 (American Type Culture Collection) was also examined for Her2/neu expression using FACS analysis. FACS staining using a Her2/neu specific antibody showed a heterogeneous positive staining of this cell line.

Antibody responses to Her2/neu were determined by Western blot analysis using recombinant Her2/neu protein (extracellular domain, ECD). Antibodies used were WT C-19 and WT 180 (Santa Cruz Biotechnology, Inc., Santa Cruz, Calif.). Western blot analysis was performed according to established protocols. As the primary antibody, sera from patients with acute myeloid leukemia as well as sera form healthy normal individuals were used in a 1:500 dilution. A donkey-polyclonal Antihuman-IgG peroxidase-conjugated second antibody (Jackson ImmunoResearch Lab, Inc.) was used in a 1:10,000 dilution. The blots were then developed by using a chemiluminescent reaction (Amersham ECL) after which they were exposed to Kodak X-OMAT™ AR film (Eastman Kodak Company, NY). The film was developed and examined.

SCID mice were inoculated with EBV-infected B-cells (see Lacerda et al., J. Exp. Med. 183:1214-1228, 1996). Lymphomas were found to be induced in all animals (a good model for lymphomas in immunocompromised organisms). The lymphomas overexpressed Her2/neu. Taken together, these data indicate that EBV-associated malignancies overexpressing Her2/neu may be treated by Her2/neu-based immunotherapy, as described herein.

### Example 3

### Priming of Her-2/neu Specific CD8+ T cells using Dendritic Cells Infected with Recombinant Adenovirus

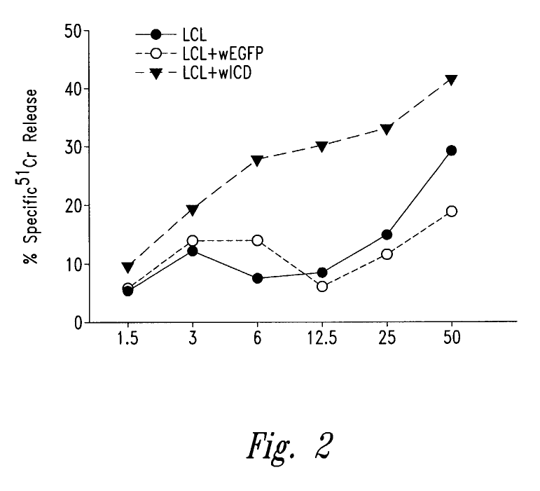

An adenovirus (AdV) vector deleted for EIA and recombinant for the intracellular domain (ICD) of Her-2/neu was constructed and used to infect dendritic cells (DC) obtained from a healthy donor. The ICD domain has been described and corresponds to a region on the Her-2/neu protein from about amino acids 676-1255. Priming cultures were initiated that contained AdV-ICD-infected DC as stimulators and autologous PBMC as responders. Prior to the first restimulation, the culture was enriched for CD8+ cells, and the CD8+-enriched population was restimulationed with AdV-ICD infected DC. Subsequent restimulations were on autologous fibroblasts transduced with a retrovirus recombinant for the ICD. Following the fourth in vitro stimulation, the resulting T cell line was tested for ICD-specific CTL activity by a standard 4 hour ⁵¹Cr-release assay. As shown in FIG. 2, the bulk T cell line contained activity specific for ICD, since the line lysed autologous B-LCL infected with vaccinia-ICD, but did not lyse C-LCL infected with vaccinia-EGFP or uninfected B-LCL targets. Each data point in FIG. 2 was the average of three measurements.

Following two more rounds of stimulation, the T cell line was tested for its ability to secrete γ-IFN in response to autologous fibroblasts expressing ICD. γ-IFN ELISPOT analysis was performed using the ICD-primed CD8+ T cell line as responders against autologous fibroblasts transduced with either ICD or EGFP. In this analysis, 2×103 fibroblasts stimulators were plated per well with 2×104 responding T cells per well, in triplicate. The average Elispot number for the triplicate wells were 344 on the ICD fibroblasts and 22 on the EGFP fibroblasts. Thus, the T cell line demonstrated ICD-specific γ-IFN secretion.

To investigate the class I restriction of the CD8+ ICD-specific T cell line, antibody blocking experiments were performed using antibodies specific for various class I molecules. Stimulators were pre-incubated either with monoclonal antibody W6/32 (HLA-A, -B and -C reactive), monoclonal antibody BB123.2 (HLA-B and -C reactive) or monoclonal antibody BB7.2 (HLA-A2 specific). T cell responses were measured using a standard overnight γ-IFN Elispot assay. Responder cells were the ICD-specific CTL line cultured in vitro for seven stimulation cycles and used at 15,000 cells per well. Stimulators were autologous fibroblasts retrovirally transduced with either ICD or EGFP and used at 2,000 cells per well. Stimulators were incubated with the indicated mAb (50 μg/mL) for 20 minutes prior to being added to the assay). The assays were performed in triplicate.

As shown in Table 2, incubation of stimulator cells with either the W6/32 or BB123.2 antibodies completely blocked recognition of the ICD-transduced fibroblasts, whereas incubation with BB7.2 had no effect on γ-IFN secretion. These results indicate that the ICD-specific activity was restricted by an HLA-B or -C allele.

TABLE 2
HLA-class I Antibody Blocking of
ICD-specific γ-IFN Secretion

Antibody Added

| Stimulators | None | BB7.2 | W6/32 | BB123.2 |
|---|---|---|---|---|
| Fibro/EGFP | 3 | 7 | 3 | 4 |
| Fibro/ICD | 167 | 213 | 4 | 5 |

An ICD-specific clone isolated from the bulk line was expanded and further characterized for its ability to recognize full-length Her-2/neu. Additionally, monoclonal antibodies specific for HLA Class I were used to examine the HLA-restriction of the clone. The experiment was a standard, overnight γ-IFN Elispot assay. Responder cells were the ICD-specific T cell clone, 17D5. Stimulators were autologous fibroblasts either untransduced or retrovirally transduced with either EGFP, ICD or full length Her2/neu (H2N). 10,000 17D5 cells and 10,000 stimulators were used per well. Antibodies were used at 25 μg/mL in the assay. The assay was performed in triplicate, and standard deviations were between 0 and +/− 18 for triplicates.

As shown in Table 3, the clone specifically recognized autologous fibroblasts transduced with ICD or full length Her-2/neu, but not untransduced fibroblasts or fibroblasts transduced with the irrelevant antigen EGFP. Furthermore, this reactivity was completely blocked by the addition of the pan-HLA Class I monoclonal antibody w6/32 and by a monoclonal antibody specific for HLA-B and -C alleles (BB123.2), but not by an antibody specific for HLA-A2 (BB7.2). These results indicate that this Her2/neu-specific clone was restricted by an HLA-B or -C allele, the same pattern of HLA restriction observed for the bulk cell line from which the clone was derived.

Further analyses indicated that the response was restricted by HLA-B4402. These analyses were performed by testing the ability of clone 17D5 to recognize a panel of allogeneic fibroblasts matched at different HLA-B and -C alleles and infected with AdV-ICD or AdV-EGFP. Autologous fibroblasts, either transduced with recombinant retroviruses or infected with recombinant AdV were used as controls.

TABLE 3
γ-IFN Elispot Assay Testing Her-2/neu Reactivity
HLA-Restriction of the lCD-specific Clone 17DS

Blocking Antibody

| Stimulators | None | W6/32 | BB123.2 | BB7.2 |
|---|---|---|---|---|
| Fibros | 0 | 0 | 0 | 0 |
| Fibro/EGFP | 0 | 0 | 1 | 0 |
| Fibro/ICD | 162 | 3 | 1 | 165 |
| Fibros-H2N | 104 | 0 | 0 | 98 |
| T cells alone | 0 | 0 | 0 | 0 |

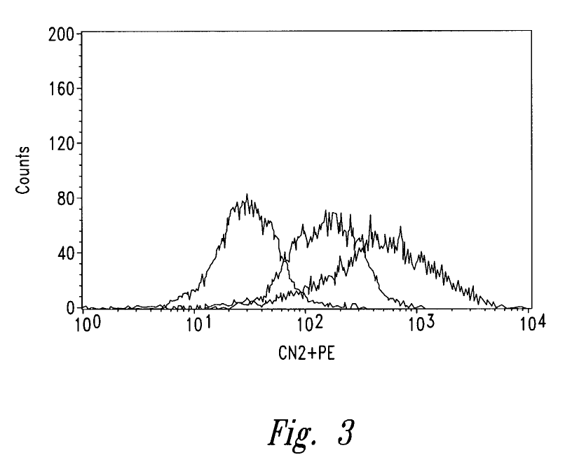

The Her-2/neu specific clone was tested for its ability to recognize human tumor cells expressing Her-2/neu. The breast carcinoma cell line MCF-7 naturally expresses low levels of Her-2/neu at the cell surface and is also HLA-b4402. Upon transduction of MCF-7 with a retrovirus recombinant for Her-2/neu, surface levels of Her-2/neu increased about 5-fold as measured by flow cytometric analysis following staining with a Her-2/neu specific monoclonal antibody. Infection of MCF-7 cells with AdV-Her-2/neu resulted in a 20-fold increase of surface Her-2/neu on the tumor cells. These results are depicted in FIG. 3.

The T cell clone secreted γ-IFN in response to MCF-7 cells infected with the adenovirus encoding Her-2/neu. The clone did not, however, appear to recognize MCF-7 cells or MCF-7 cells transduced with the retrovirus expressing Her-2/neu. Since the clone does recognize human fibroblasts transduced with either ICD or Her2/neu, and since the transduced fibroblasts express similar levels of protein as the transduced MCF-7 cells, it is unlikely that this result is due solely to levels of expression of the antigen.

### Example 4

### Identification of an HLA-B44-Restricted, Naturally Processed Epitope of Her-2/Neu

This example describes the characterization of the epitope recognized by one of the T cell clones described above, 17D5. This clone recognized APC expressing the ICD or full-length Her-2/neu protein. The HLA-restriction element for the clone was determined to be HLA-B4402 by using a panel of allogenic cell lines matched at various HLA alleles with the T cell clone as APC in gamma interferon Elispot assays. This was confirmed by transduction of HLA-B44-negative, Her-2/neu-positive APC with a B4402-recombinant retrovirus to confer recognition. The region of the ICD recognized by the clone was narrowed by using recombinant retroviruses expressing a series of five fragments of the ICD to transduce B44+ APC. Recognition (as demonstrated by gamma interferon release) by the clone of two of these fragments indicated that the epitope was contained within a 235 amino acid fragment beginning at position 975 in the Her-2/neu sequence. Predicted B44-binding 9mer and 10mer peptides from within this fragment were chosen and synthesized. Of the 13 peptides synthesized, one was recognized by the clone and determined to be the epitope. This has been demonstrated by gamma interferon release and TNF-alpha release assays. The sequence of this naturally processed Her-2/neu epitope is: EEYLVPQQGF (SEQ ID NO: 3), position 1021-1030 in the Her-2/neu protein sequence.

### Example 5

### T Cell Clone Specific for Her-2/neu Recognizes Human Tumor Cells

A T cell clone specific for Her-2/neu was derived by priming in vitro with autologous dendritic cells infected with an adenovirus recombinant for the ICD of Her-2/neu, as described in the Examples above. To determine the ability of this T cell clone to recognize human tumors that endogenously express Her-2/neu, the following experiments were performed.

The human tumor cell lines SKBR3 (breast carcinoma) and SKOV3 (ovarian carcinoma) both overexpress Her-2/neu. The human tumor cell lines HCT-116 (colon carcinoma) and MCF-7 (breast carcinoma) express very little or no Her-2/neu protein. Of these tumors, only MCF-7 naturally expresses HLA-B4402 (the restriction allele for this clone) as indicated by HLA typing. A retrovirus recombinant for HLA-B4402 was used to transduce SKOV3, SKBR3, and HCT-116 tumor cell lines. Flow cytometric analysis was performed to examine HLA class I, HLA-B44, and Her-2/neu expression on parental and transduced tumor cell lines and fibroblast cell line controls. Tumor cell lines or fibroblast cell lines were stained with the following FITC-labeled monoclonal antibodies: IgG (Becton Dickinson, negative control); anti-HLA class I antibody (Sigma); anti-Bw4 antibody, which binds a subgroup of HLA-B molecules, including HLA-B44 (One Lambda); anti-Her-2/neu antibody CN2, (Ab2 from Oncogene Sciences). Samples were fixed and analyzed by flow cytometry.

Results demonstrated that all cell lines tested expressed HLA-class I at the cell surface, as expected. The autologous fibroblasts and MCF-7 tumor cells, but not the parental tumor cell lines SKBR3, SKOV3, or HCT-116, expressed HLA-B44. Following transduction with HLA-B44 retrovirus, each tumor cell line gained expression of HLA-B44. As expected, SKBR3 and SKOV3 tumor cell lines both expressed Her-2/neu at the cell surface, and levels were comparable to the amount of Her-2/neu expressed by the autologous fibroblasts retrovirally transduced with Her-2/neu. In contrast, MCF-7, HCT-116, and the non-transduced fibroblasts expressed very little or no Her-2/neu at the cell surface. MCF-7 cells transduced with retrovirus recombinant for Her-2/neu expressed levels of Her-2/neu that were comparable to those expressed by SKBR3 and SKOV3.

The ability of the Her-2/neu-specific CTL clone (clone 17D5) to recognize the above cell lines was tested in IFNγELISAs and TNFα bioassays. Table 4 depicts the results of an IFNγ ELISA. Clone 17D5 specifically secreted IFNγ in response to autologous HLA-B4402-positive fibroblasts transduced to express Her-2/neu. Importantly, clone 17D5 specifically secreted IFNγ in response to SKBR3 and SKOV3 tumor cells transduced with HLA-B4402, but not to the HLA-B4402-negative parental, or control transduced tumor cell lines. Clone 17D5 did not recognize the HCT-116 tumor cell line transduced with HLA-B4402. This result was expected since HCT-116 cells express only very low levels of Her-2/neu. 17D5 also did not recognize the breast tumor cell line MCF-7 or MCF-7 transduced with Her-2/neu. These results are most likely explained by insufficient levels of HLA-B4402 expressed by MCF-7, since the levels of Her-2/neu on MCF-7 cells transduced with the Her-2/neu retrovirus are similar to the levels of Her-2/neu on the corresponding transduced fibroblasts. (This could be overcome by expressing very high levels of Her-2/neu in MCF-7 cells via infection with a Her-2/neu-recombinant adenovirus.)

TABLE 4
IFN_{?} ELISA Demonstrating Tumor
Recognition by ICD-specific T Cell Clone 17D5¹

| Stimulators | Ave O.D.² |
|---|---|
| FIB | 0.09 |
| H2N Fib | 1.14 |
| HCT116-EGFP | 0.11 |
| HCT116-B44 | 0.10 |
| SKBR3 | 0.08 |
| SKBR3-B44 | 1.81 |
| SKOV3-EGFP | 0.10 |
| SKOV3-B44 | 1.22 |
| MCF7 | 0.09 |
| MCF7-H2N | 0.09 |
| T cells only | 0.10 |
| Media only | 0.1 |
| ¹A standard IFNγ ELISA was performed using 24 hour supernatants obtained from incubating clone 17D5 T cells with the indicated stimulators, or media alone. 17D5 T cells and stimulators were each used at 10,000 cells/well in the assay. Assay was performed in triplicate in 96-well plates. Supernatants from stimulators incubated without T cells were included as controls, with none of the values were greater than background (data not shown). | |
| # Following development of the ELISA, O.D. was read at 450 nm, using 570 nm as a reference. | |

²Data shown are averages of O.D. readings for triplicate wells.

The results of TNFα bioassay were consistent with the results of the IFN-γELISA: clone 17D5 specifically secreted TNFα in response to both SKBR3 and SKOV3 when these cell lines were transduced with the HLA-B4402-expressing retroviral construct (Table 5).

TABLE 5
TNFa Bioassay Demonstrating Tumor Recognition
by T Cell Clone 17D5¹

| T Cells + APC | Ave O.D.² |
|---|---|
| FIB | 0.902 |
| H2N FIB | 0.327 |
| HCT116-EGFP | 1.036 |
| HCT116-B44 | 0.978 |
| SKBR3 | 1.057 |
| SKBR3-B44 | 0.359 |
| SKOV3-EGFP | 1.070 |
| SKOV3-B44 | 0.381 |
| MCF7 | 1.073 |
| MCF7-H2N | 0.878 |
| T cells only | 0.995 |
| Media only | 1.038 |
| ¹Clone 17D5 T cells (10,000 cells/well) were incubated with the indicated APC (10,000 cells/well) or in media alone in 96-well plates in triplicate. Four hour supernatants were harvested and added to the TNFα-sensitive cell line WEHI, plated at 30,000 cells/well in a 96-well plate. WEHI cells were incubated overnight with the supernatants, and alomar blue was added to 1/10final volume per well. O.D. 570 nm-630 nm was read at 7 hours and 24 hours after addition of Alomar blue. | |
| #Results shown are from the 24 hour timepoint. | |

²O.D. values are averages of triplicate wells and indicate the relative viability of the TNFα-sensitive WEHI cells, with lower values being indicative of increased cell death, therefore increased TNFα secretion.

The above results are significant because they demonstrate that CD8+ T cells primed in vitro using an ICD-recombinant adenovirus are capable of recognizing human tumor cells that over express Her-2/neu. Twenty to forty percent of human breast carcinomas, as well as a proportion of carcinomas of the ovary, lung, and colon over express Her-2/neu. These data support the use of ICD as a vaccine for Her-2/neu-positive tumors.

Sequence Identifiers:

SEQ ID NO: 1 sets forth a DNA sequence encoding the Her-2/neu protein.

SEQ ID NO: 2 sets forth the amino acid sequence for the Her-2/neu protein.

SEQ ID NO: 3 sets forth the polypeptide sequence for a naturally processed epitope of Her-2/neu corresponding to amino acids 1021-1030 of the Her-2/neu protein.

All publications and patent applications mentioned in this specification are herein incorporated by reference to the same extent as if each individual publication or patent application was specifically and individually incorporated by reference.

From the foregoing, it will be evident that, although specific embodiments of the invention have been described herein for purposes of illustration, various modifications may be made without deviating from the spirit and scope of the invention. Accordingly, the present invention is not limited except as by the appended claims.

What is claimed:

1. A method for inhibiting the development of a hematological malignancy in a patient, comprising administering to a patient an effective amount of a polypeptide comprising at least an immunogenic portion of Her2/neu, or a polynucleotide encoding said polypeptide, wherein said hematological malignancy is selected from the group consisting of AML, CML, CLL, MDS, myelomas, Hodgkin lymphomas and non-Hodgkin lymphomas, and thereby inhibiting the development of the hematological malignancy in the patient.

2. The method of claim 1, wherein said immunogenic portion of Her2/neu comprises an amino acid sequence consisting essentially of SEQ ID NO: 3.

3. A method for inhibiting the development of a hematological malignancy in a patient, comprising administering to a patient an effective amount of antigen-presenting cells that express a polypeptide comprising at least an immunogenic portion of Her2/neu, wherein said hematological malignancy is selected from the group consisting of AML, CML, CLL, MDS, myelomas, Hodgkin lymphomas and non-Hodgkin lymphomas, and thereby inhibiting the development of the hematological malignancy in the patient.

4. The method of claim 3, wherein said immunogenic portion of Her2/neu comprises an amino acid sequence consisting essentially of SEQ ID NO: 3.

5. A method according to claim 3, wherein the antigen-presenting cells are dendritic cells.

6. A method for inhibiting the development of a hematological malignancy in a patient, comprising the steps of:
(a) incubating CD4⁺ and/or CD8+T cells isolated from a patient with at least one component selected from the group consisting of:
(i) a polypeptide comprising at least an immunogenic portion of Her2/neu;
(ii) a polynucleotide encoding such a polypeptide; and
(iii) an antigen-presenting cell that expresses such a polypeptide; such that T cells proliferate; and
(b) administering to the patient an effective amount of the proliferated T cells, and thereby inhibiting the development of a hematological malignancy in the patient.

7. The method of claim 6, wherein said immunogenic portion of Her2/neu comprises an amino acid sequence consisting essentially of SEQ ID NO: 3.

8. A method according to claim 6, wherein the hematological malignancy is selected from the group consisting of AML, CML, CLL, MDS, myelomas, Hodgkin lymphomas and non-Hodgkin lymphomas.


SEQUENCE LISTING (5 sequences)

SEQ ID NO: 1 - DNA, 3768 bp, Homo sapien
  CDS (1)...(3765)
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtga

SEQ ID NO: 2 - protein, 1255 aa, Homo sapien [sequence omitted]

SEQ ID NO: 3 - protein, 10 aa, Homo sapiens
EEYLVPQQGF

SEQ ID NO: 4 - DNA, 48 bp, Artificial Sequence
  Oligonucleotide Primer for for PCR recovery of HER-2/neu polypeptide
tctggcgcgctggatgacgatgacaagaaacgacggcagcagaagatc

SEQ ID NO: 5 - DNA, 39 bp, Artificial Sequence
  Oligonucleotide Primer for PCR recovery of HER-2/neu polypeptide
tgaattctcgagtcattacactggcacgtccagacccag